(19) United States
(12) Patent Application Publication
(10) Pub. No.: US 2008/0269657 A1
(43) Pub. Date: Oct. 30, 2008

(54) ADHESIVE OVERBANDAGE

(75) Inventors: Allen Brenneman, Goshen, IN (US); Richard G. Sass, Portland, OR (US); W. Kenneth Ward, Portland, OR (US); Jennifer Matson, Portland, OR (US); Dennis Slomski, Tualatin, OR (US); Robert Bruce, Beaverton, OR (US); Leslie Wood, Wilsonville, OR (US); Isaac Federiuk, Tigard, OR (US)

Correspondence Address: SCHWABE, WILLIAMSON & WYATT, P.C.;PACWEST CENTER, SUITE 1900, 1211 SW FIFTH AVENUE, PORTLAND, OR, 97204, US

(73) Assignee: ISENSE CORPORATION, Wilsonville, OR (US)

(21) Appl. No.: 12/108,874

(22) Filed: Apr. 24, 2008

Related U.S. Application Data
(60) Provisional application No. 60/914,299, filed on Apr. 26, 2007.

(51) Int. Cl. A61B 19/02; A61F 13/02

(52) U.S. Cl. 602/41; 128/869

(57) ABSTRACT

Embodiments of the present invention provide an adhesive overbandage. In an embodiment, there is provided an adhesive overbandage for use with a biosensor. An exemplary overbandage comprises a frame having an upper surface, a lower surface, an outer boundary and an inner boundary, wherein the inner boundary defines an interior opening configured to receive a monitoring device, adhesive disposed on the lower surface of the frame, and a release liner removably coupled to the adhesive.

### CROSS-REFERENCE TO RELATED APPLICATIONS

The present application claims priority to U.S. Provisional Patent Application No. 60/914,299, filed Apr. 26, 2007, entitled “Adhesive Overbandage,” the entire disclosure of which is hereby incorporated by reference in its entirety.

### TECHNICAL FIELD

Embodiments of the present invention relate to the field of bandages, and, more specifically, to an adhesive overbandage.

### BACKGROUND

Perspiration may be a major problem for patients who are wearing a device that is taped or adhered to the skin. Water may build up beneath the dressing/bandage and lift it off the skin because of perspiration, and/or due to other sources of water or other substances, etc. In the case of a biosensor that is kept in place or anchored by a bandage, dislodgement of the bandage may lead to dislodgement (full or partial) of the sensor itself. The degree of the problems may increase in some users with prolonged use.

### BRIEF DESCRIPTION OF THE DRAWINGS

Embodiments of the present invention will be readily understood by the following detailed description in conjunction with the accompanying drawings. Embodiments of the invention are illustrated by way of example and not by way of limitation in the figures of the accompanying drawings.

FIG. 1 illustrates a top view of an exemplary rectangular overbandage in accordance with an embodiment of the present invention;

FIG. 2 illustrates a top view of an exemplary oval overbandage in accordance with an embodiment of the present invention;

FIG. 3 illustrates a cross-sectional view of an exemplary multi-layered overbandage in accordance with an embodiment of the present invention;

FIG. 4 illustrates a cross-sectional view of an overbandage in accordance with an embodiment of the present invention;

FIGS. 5A and 5B illustrate an overbandage coupled to a two-part release liner in accordance with an embodiment of the present invention;

FIGS. 6A and 6B illustrate the two-part release liner from FIGS. 5A and 5B in an unfolded and separated state in accordance with an embodiment of the present invention;

FIG. 7 illustrates an overbandage just prior to application with a monitoring device in accordance with an embodiment of the present invention;

FIG. 8 illustrates an overbandage applied over and around a monitoring device in accordance with an embodiment of the present invention;

FIGS. 9A, 9B, and 9C illustrate an overbandage guide in accordance with an embodiment of the present invention; and

FIGS. 10A, 10B, and 10C illustrate an overbandage placement device in accordance with an embodiment of the present invention.

### DETAILED DESCRIPTION OF EMBODIMENTS OF THE INVENTION

In the following detailed description, reference is made to the accompanying drawings which form a part hereof, and in which are shown by way of illustration embodiments in which the invention may be practiced. It is to be understood that other embodiments may be utilized and structural or logical changes may be made without departing from the scope of the present invention. Therefore, the following detailed description is not to be taken in a limiting sense, and the scope of embodiments in accordance with the present invention is defined by the appended claims and their equivalents.

Various operations may be described as multiple discrete operations in turn, in a manner that may be helpful in understanding embodiments of the present invention; however, the order of description should not be construed to imply that these operations are order dependent.

The description may use perspective-based descriptions such as up/down, back/front, and top/bottom. Such descriptions are merely used to facilitate the discussion and are not intended to restrict the application of embodiments of the present invention.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

For the purposes of the description, a phrase in the form “A/B” or in the form “A and/or B” means “(A), (B), or (A and B)”. For the purposes of the description, a phrase in the form “at least one of A, B, and C” means “(A), (B), (C), (A and B), (A and C), (B and C), or (A, B and C)”. For the purposes of the description, a phrase in the form “(A)B” means “(B) or (AB)” that is, A is an optional element.

The description may use the phrases “in an embodiment,” or “in embodiments,” which may each refer to one or more of the same or different embodiments. Furthermore, the terms “comprising,” “including,” “having,” and the like, as used with respect to embodiments of the present invention, are synonymous.

Embodiments of the present invention provide an adhesive overbandage. In an embodiment, there is provided an adhesive overbandage for use with a biosensor.

For the purposes of the present invention, the term “overbandage” refers to a bandage that may be applied over a device that is resident on the skin or already adhered to skin using adhesive and/or another bandage. An overbandage has particular benefits in that it may be applied over an existing device/bandage to extend the time that the existing device/bandage adheres to or remains on the skin.

During prolonged use of adhesives or adhesive bandages on skin, the strength or tackiness of the adhesive may decline and the secured device and/or bandage may begin to peel away from the skin. This may be caused, for example, by the user sweating, by contact with water from showering, swimming, etc., by contact with skin oils and/or sebum, by contact with dirt and other substances, etc. In addition, a device and/or bandage may begin to peel away from the skin prior to the desired time for removal as a result of the individual picking at the bandage or bumping or catching the device/bandage on clothing, furniture, etc. Thus, there are numerous situations that may cause the adhesive on a device or bandage to become dislodged giving rise to the need for an overbandage to further secure the device.

As an example, a continuous glucose monitoring device may include a sensor coupled to an on-skin monitoring unit. The sensor may be inserted into skin of a user, and a signal indicative of the amount of glucose in the sensed tissue/fluid of the user may be communicated to electronic circuitry in the on-skin unit. In an embodiment, such a continuous glucose monitoring device may be adhered to the skin with an adhesive bandage or adhesive which may be applied directly to the underside of the device and/or on the skin of the user. Such a device may have a prescribed usage period of, for example, 3, 5, 7 days or more before which the sensor needs to be replaced. Due to the relative cost difference of the goods, and the relative difficulty of replacement, if the device or adhesive becomes partially dislodged prior to the expiration of the usage period (3, 5, 7 days or more), it may, in embodiments, be more cost effective and/or more desirable to utilize an overbandage to resecure the device on the skin as opposed to removing the device/sensor and starting anew with a new sensor and/or with new adhesive.

Thus, in an embodiment, there is provided an overbandage, comprising a frame having an upper surface, a lower surface, an outer boundary and an inner boundary, wherein the inner boundary defines an interior opening configured to receive a monitoring device, adhesive disposed on the lower surface of the frame, and a release liner removably coupled to the adhesive.

FIG. 1 illustrates a top view of an exemplary overbandage 100 in accordance with an embodiment of the present invention. Overbandage 100 is shown with rectangular frame 102 and interior 104. In an embodiment, interior 104 may be absent any material. In an alternative embodiment, interior 104 may comprise a material, such as a non-adhesive material.

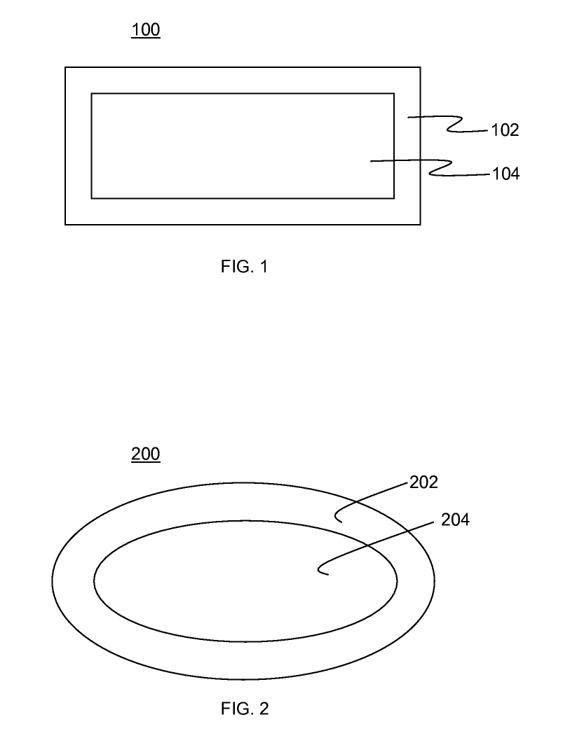

FIG. 2 illustrates a top view of an exemplary overbandage 200 in accordance with an embodiment of the present invention. Overbandage 200 is shown with oval frame 202 and interior 204. In an embodiment, interior 204 may be absent any material. In an alternative embodiment, interior 204 may comprise a non-adhesive material. In another embodiment, interior 204 may comprise an adhesive or partially adhesive material.

While FIGS. 1 and 2 illustrate two exemplary shapes of overbandages in accordance with embodiments of the present invention, other shapes may be utilized such as squared, rounded, polygonal, regular, irregular, etc. In an embodiment, while FIGS. 1 and 2 show both interior and exterior boundaries of the frame matching, the shapes of the boundaries may differ, for example, such that an oval shaped overbandage may have a rectangular shaped interior, etc.

The width of the frame of an overbandage (such as frames 102 and 202 shown in FIGS. 1 and 2), may be any suitable width, but preferably are sufficiently wide to provide adequate adhesion. In an embodiment, a frame is wide enough to contact both an underlying adhesive bandage/patch and the skin of the individual while adhering sufficiently to both.

In an embodiment, a frame may be approximately 0.25 inch to approximately 2 inches wide, or larger. In an embodiment, an interior (such as interiors 104 and 204) may have width and length dimensions of approximately 1-2 inches by 1-2 inches. These dimensions are merely exemplary and may be adjusted as desired for use with various devices. In an embodiment, an interior may be sized and shaped specifically to match the shape of a device with which the overbandage is intended to be associated.

In an embodiment, a frame may be single layered or multilayered. In an embodiment, a frame may be a plastic, foam, or fabric-based material/tape with adhesive on one side. In an embodiment, a frame may be a film-based or polyurethane-based bandage material. In an embodiment, a frame may be transparent, translucent, or opaque, and, in an embodiment, may be made in one or more of a variety of colors. In an embodiment, a frame may provide wicking of water, such as sweat, and/or breathability to allow passage of water vapor.

In embodiments, suitable adhesives include medical adhesives, acrylics, cyanoacrylates, methacrylates, fibrin glue, etc. In an embodiment, an adhesive having a relatively high vapor transmission rate may be utilized to enhance the movement of water and/or water vapor away from the skin.

In an embodiment, an overbandage may be breathable (i.e., it may allow water vapor to release toward the outside but not allow liquid water to penetrate inward). In an embodiment, a breathable overbandage may be constructed from one or more breathable materials in one or more layers. In another embodiment, regardless of the material(s) selected, a breathable overbandage may be constructed, for example, by creating small pores (by using a laser or other means) in the frame material. In an embodiment, suitable pores may have a diameter of approximately 100 Angstroms to approximately 5 microns. In embodiments, the pores may be interconnected, or may be separate.

In an embodiment, water vapor release by the overbandage may be enhanced by utilizing a material that wicks moisture away from the skin. In an embodiment, a separate wicking layer (e.g. a nonwoven fabric layer) may be applied above or, preferably, below a breathable layer.

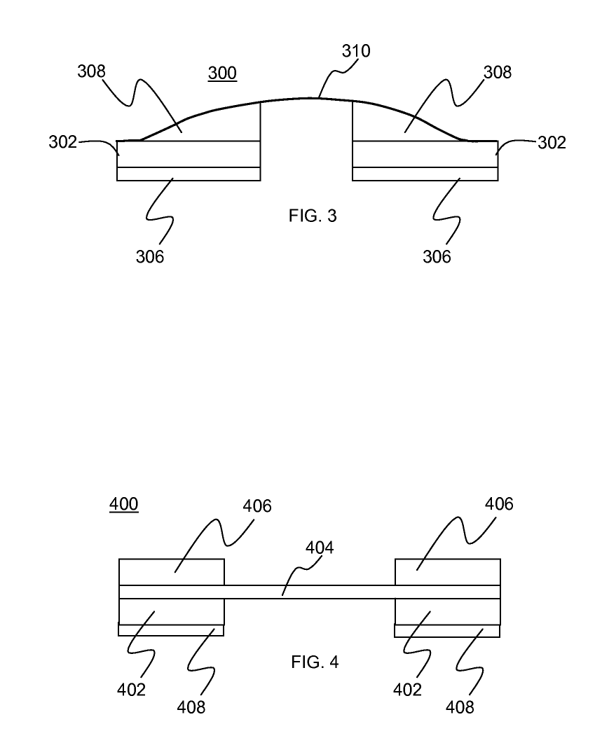

FIG. 3 illustrates a cross-sectional view of an exemplary multi-layered overbandage 300 in accordance with an embodiment of the present invention. Overbandage 300 is shown with frame 302. In an embodiment, frame 302 may be a supporting layer, or may be a wicking or breathable layer. Disposed below frame 302 is shown adhesive 306, which may be provided as a uniform sheet, patterned, channeled, etc. A wicking layer 308 is shown disposed above frame 302. In an embodiment, a single wicking layer may be provided instead of both frame 302 and layer 308. Above wicking layer 308 is shown breathable layer 310, such as a breathable film. Breathable layer 310 may be attached to the underlying features at one or more of a variety of locations, and, in an embodiment, may cover some or all of overbandage 300. In an embodiment, wicking layer 308, frame 302, and breathable layer 310 may be constructed from the same or different materials. The arrangement of FIG. 3 is merely exemplary and such an overbandage may be constructed with more or less layers, in various configurations, as desired.

In embodiments, any suitable material may be used for a breathable and/or wicking layer, such as a polymer, for example polyethylene terephthalate, semi-crystalline polyamides, polyimides such as Nylon 6 or Nylon 66, polyolefins such as polyethylene and polypropylene, polymethylpentene, polyisobutylene, polyolefin copolymers, polyester copolymers, fluoropolymers, poly vinyl acetate, poly vinyl alcohol, polyethylene oxide, functionalized polyolefins, ethylene vinyl acetate copolymers, polytetrafluoroethylene, or amorphous thermoplastic materials to include polycarbonates, polyacrylics, polymethacrylics, polybutadiene, polyisoprene, polychloroprene, random and block copolymers of styrene and dienes, butyl rubber, ethylene-propylene-diene monomer rubber, natural rubber, ethylene-propylene rubber, and mixtures thereof. Other examples of suitable materials may include polyvinyl chloride, thermoplastic polyurethanes, aromatic epoxies, amorphous polyesters, amorphous polyamides, acrylonitrile-butadiene-styrene (ABS) copolymers, polyphenylene oxide alloys, high impact polystyrene, polystyrene copolymers, fluorinated elastomers, polydimethyl siloxane, polyetherimides, methacrylic acid-polyethylene copolymers, impact-modified polyolefins, or amorphous fluoropolymers.

In an embodiment as mentioned above, an overbandage may have an interior which is absent any material, or, in an embodiment, an overbandage may be provided with material spanning the interior.

FIG. 4 illustrates a cross-sectional view of an overbandage 400. Overbandage 400 shows two portions of frame 402. Overbandage 400 also shows material 404 spanning the interior opening defined by frame 402. In an embodiment, additional material 406 may be provided and arranged, for example, as a frame as shown. Material 406 may be the same material or different from that of frame 402. In an embodiment, material 406 may be provided to “sandwich” or further secure material 404. While FIG. 4 shows the edges of frame 402, material 404, and material 406 aligned, other arrangements may be provided as well, for example in which material 404 does not extend to the outer edge of frame 402. In an embodiment, material 404 may be secured in a desired location on frame 402 (and potentially on material 406) using one or more of a variety of attachment mechanisms, including adhesive. In an embodiment, frame 402 and material 404 may be utilized without additional material 306. In an embodiment, adhesive 408 may be disposed on frame 402 to adhere overbandage 400 to skin and over a device/bandage.

In an embodiment, material 404 may be waterproof, such as a waterproof film. In an embodiment, material 404 may be selected to keep microorganisms from penetrating through the bandage, and may, in an embodiment, allow water vapor to escape. In an embodiment, material 404 may prevent water in any form in any significant quantity to cross the bandage in a direction toward the underlying device or skin. In an embodiment, material 404 may be transparent, translucent, or opaque, and/or may be made in one or more of a variety of colors. In an embodiment, material 404 may be entirely non-adhesive, or at least non-adhesive in the region that spans the interior defined by frame 402. In another embodiment, material 404 may include adhesive on the surface that contacts the underlying device to help secure overbandage 400 to the underlying device. In such an embodiment, a suitable adhesive may be one that allows overbandage 400 to be relatively easily detached from the underlying device. In an embodiment, material 404 may be sufficiently flexible to stretch without breaking when placed over and around an on-skin unit/device. In an embodiment, material 404 may be flexible or elastic to permit material 404 to fit relatively snug to an associated device and to permit the placement of overbandage 400 in a proper secure position on the underlying bandage/device and skin of the user.

In an embodiment, a frame may be provided with adhesive on the underside or lower surface (skin-contact surface). In an embodiment, the adhesive on an overbandage may be covered by a release liner that may be easily removed just prior to adhering the overbandage to the skin. In an embodiment, a release liner may be provided in a variety of shapes and may be in one or multiple parts, such as including tabs or wings, to enable attachment of the overbandage with minimal difficulty for the user.

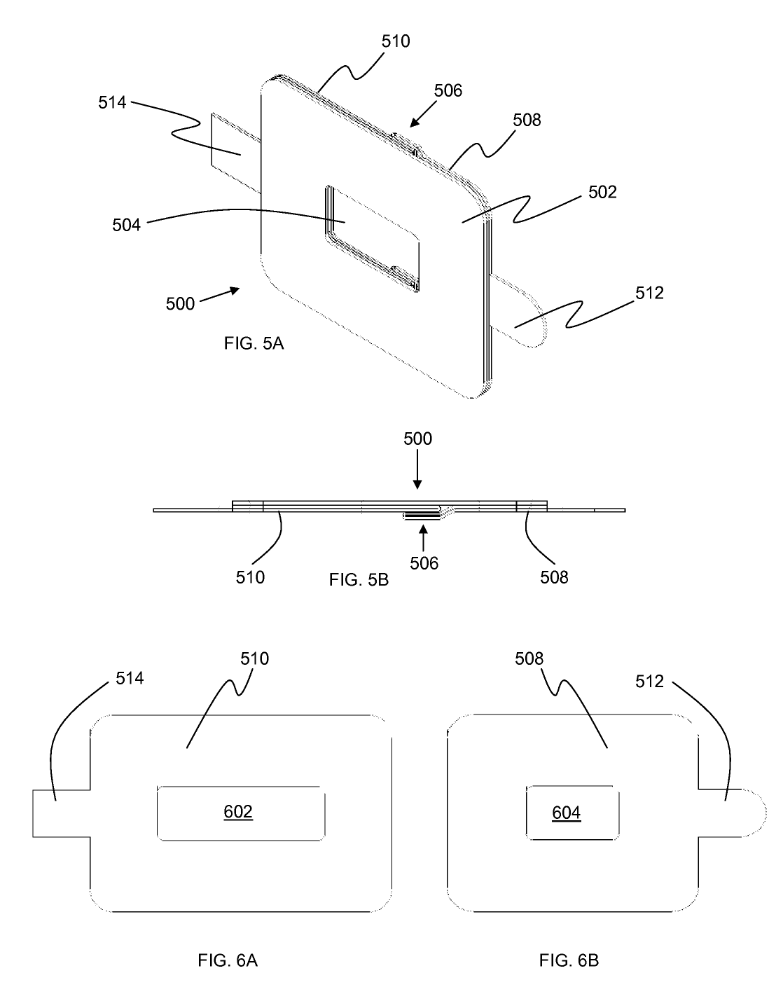

FIGS. 5A and 5B illustrate an overbandage 500 coupled to an exemplary two-part release liner 506 in accordance with an embodiment of the present invention. Overbandage 500 has a frame 502 and interior 504. Release liner 506 has two parts 508, 510. In an embodiment, release liner parts 508, 510 may each be folded as shown. In an embodiment, the surfaces of each of parts 508, 510 that contact each other or contact another surface of another of parts 508, 510 may be provided with adhesive, as desired, to maintain the folded arrangement of parts 508, 510. As shown, parts 508, 510 may overlap each other. Parts 508, 510 have tabs 512, 514 respectively. Tabs 512, 514 provide a location to be grasped by a user to remove release liner parts 508, 510. In operation, in an embodiment, a user may pull tab 512 exposing adhesive on a portion of the underside of overbandage 500. A user may also pull tab 514, simultaneously with, before, or after pulling tab 514 thus exposing adhesive on the other portion of the underside of overbandage 500. In an embodiment, the overlapping arrangement of parts 508, 510 may suggest an order of removal, namely the outermost part (part 508) being removed first.

In embodiments, overbandage 500 may be placed over an on-skin unit before or after removal of release liner 506. In an embodiment, providing a release liner that does not cover or encircle interior 504 allows overbandage 500 to be placed over an on-skin unit and for subsequent removal of release liner 506 thus reducing the extent to which the adhesive of overbandage 500 is disturbed by the user or impacted by environmental conditions (dirt, debris, water, etc.).

In other embodiments, a release liner may be provided with more or less parts that may or may not overlap. In an embodiment, tabs may or may not be provided, and, if provided, such tabs may be of any suitable size, shape, color, etc. In an embodiment, tabs may be color coded and/or numbered to indicate the recommended order of removing the various release liner parts.

FIGS. 6A and 6B illustrate the two-part release liner of FIGS. 5A and 5B in an unfolded and separated state in accordance with an embodiment of the present invention. FIG. 6A illustrates an unfolded release liner part 510 having a tab 514 and open interior 602. FIG. 6B illustrates an unfolded release liner part 508 having a tab 512 and open interior 604.

In an embodiment, an overbandage may be provided individually in a peel pouch, sterilized, for example, by ethylene oxide or another sterilizing agent.

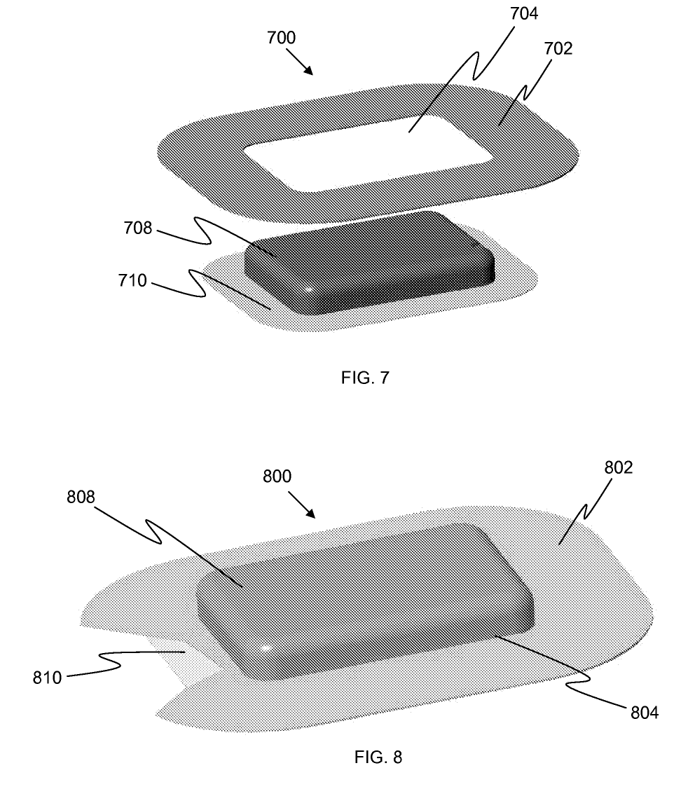

FIG. 7 illustrates an overbandage 700 just prior to application with a monitoring device 708. Overbandage 700 is shown with rectangular frame 702 and interior 704. In an embodiment, interior 704 may be absent any material. In an alternative embodiment, interior 704 may comprise a non-adhesive, adhesive, or partially adhesive material. Overbandage 700 is poised above and ready to be applied over monitoring device 708 and adhesive bandage/patch 710. In an embodiment, an exemplary monitoring device is a continuous glucose monitoring device. In an embodiment, adhesive bandage/patch 710 may cover the entire underside of monitoring device 708. In another embodiment, adhesive bandage/patch 710 may cover only a portion of the underside of monitoring device, for example to provide a defined opening for exit of an associated sensor. In an embodiment in which adhesive bandage/patch 710 covers the entire underside of monitoring device 708, an associated sensor may exit through the end or side of the on-skin unit, or, in an embodiment, such a sensor may have sufficient strength to pierce the adhesive bandage/patch or a separate material (foil, film, etc.) disposed in the desired exit location for the sensor.

In an embodiment when an overbandage is applied to a device on the skin of a user, additional adhesive may be applied to the underside of the overbandage and/or to the contact surfaces on the device, existing bandage/patch, and/or skin to increase the adhesive qualities of the components. In an embodiment, prior to applying the overbandage, the skin or surfaces of the device/bandage may be cleaned or prepped with a suitable solution or compound to sterilize and/or pre-treat the area.

FIG. 8 illustrates an overbandage 800 applied over and around an on-skin monitoring device 808 in accordance with an embodiment of the present invention. Overbandage 800 has frame 802 and interior opening 804. A portion of frame 802 has been cut away for illustration purposes to show the underlying adhesive bandage/patch 810. In an embodiment, overbandage 800 may have an interior opening 804 that is larger than the outer dimension of on-skin monitoring device 808 such that a portion of adhesive bandage/patch 810 may be visible around on-skin monitoring device 808 in/through interior opening 804.

Thus, in an embodiment, there is provided a method of further securing a monitoring device to skin, comprising placing an overbandage over and around the monitoring device, the monitoring device being at least partially secured to the skin with a primary bandage, the overbandage comprising a frame having an upper surface, a lower surface, an outer boundary and an inner boundary, wherein the inner boundary defines an interior opening, adhesive disposed on the lower surface of the frame, and a release liner removably coupled to the adhesive, and removing the release liner to expose the adhesive and adhere the overbandage to the skin and over at least part of the primary bandage.

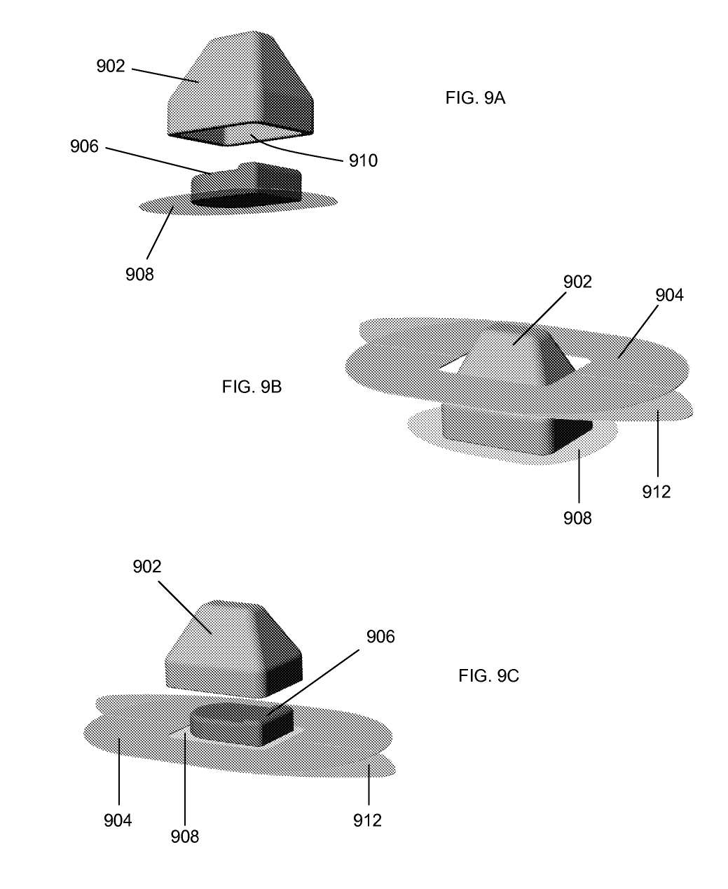

FIGS. 9A, 9B, and 9C illustrate an overbandage guide 902 in accordance with an embodiment of the present invention. FIG. 9A shows a view of guide 902 prior to engagement with on-skin monitoring device 906 and primary bandage/patch 908. FIG. 9B illustrates a view of an overbandage 904 just prior to application and following the direction of guide 902. FIG. 9C shows a view of guide 902 after being disengaged with on-skin monitoring device 906.

Guide 902 may be used to help a user properly align overbandage 904 with on-skin monitoring device 906 and primary bandage/patch 908. Guide 902 has an opening 910 in the base of the housing of guide 902 that may be configured to mate with the size and/or shape of one or more on-skin monitoring devices, such as on-skin monitoring device 906. In an embodiment, the orientation of guide 902 may be specific to the size and/or shape of an on-skin monitoring device, such that guide 902 will fit in one orientation, but not in another. In an embodiment, additional markings, colors, etc. on guide 902, overbandage 904, primary bandage/patch 908, and/or on-skin monitoring device 906 may be used to orient and/or properly align the components.

FIGS. 9B and 9C also show overbandage 904 with release liner 912. Release liner 912 may be removed from overbandage 904 at any point during the placement process. In an embodiment, release liner 912 may be removed after overbandage 904 has been placed on/around on-skin monitoring device 906 so that the adhesive surface of overbandage 904 has less opportunity to contact dirt, debris, water, etc. or to accidentally adhere to the surface of guide 902. Release liner 912 may be removed before or after disengagement of guide 902 from monitoring device 906.

In an embodiment, guide 902 may be reused a plurality of times, or, if desired, may be disposable.

In an embodiment, there is provided an overbandage guide, comprising a housing having an opening configured to mate with an on-skin monitoring device, the housing having a base, and the base having a size and/or shape configured to correspond to an opening of an overbandage to guide the orientation and/or direction of the overbandage over the housing and onto skin of a user or on and/or around a component on the skin of the user.

For the purposes of describing embodiments, the term “configured to correspond” means that the size and/or shape of various components are matched or similar or that one component does not substantially hinder the placement and/or function of an associated component.

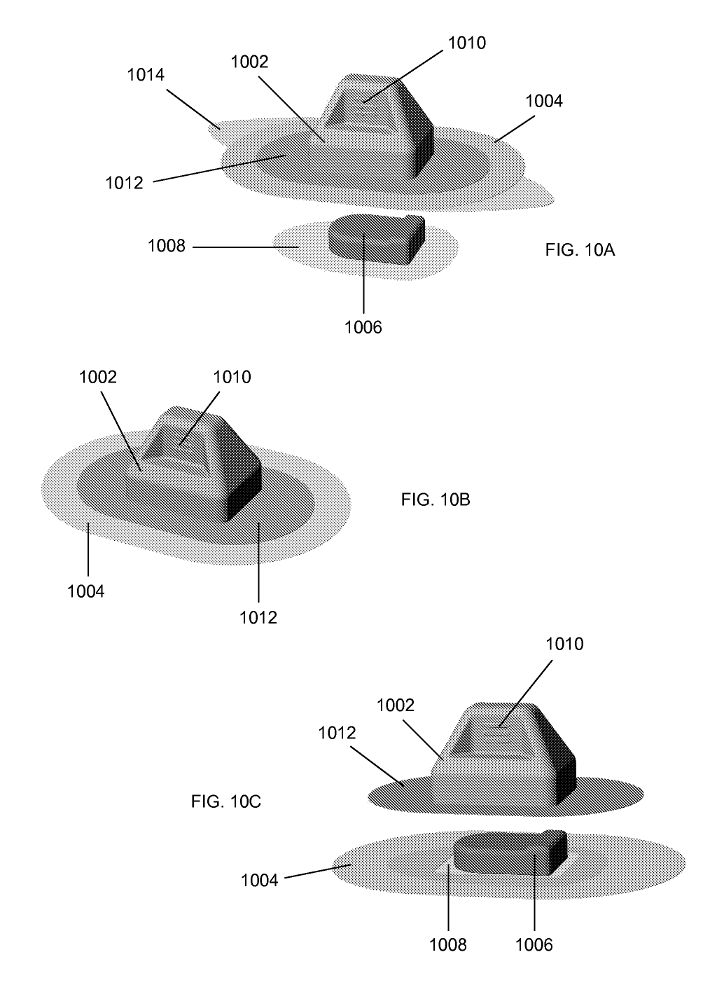

FIGS. 10A, 10B, and 10C illustrate an overbandage placement device 1002 in accordance with an embodiment of the present invention. FIG. 10A shows placement guide 1002 associated with overbandage 1004 prior to placement on/around on-skin monitoring device 1006 and primary bandage/patch 1008. In an embodiment, placement guide 1002 may have one or more features, such as grip 1010, for grasping by a user. In an embodiment, placement device 1002 may have an integrated support 1012 for removably coupling with and structurally supporting overbandage 1004. Support 1012 may have a low tack adhesive on the overbandage contact surface to provide a coupling with overbandage 1004. Alternatively, placement guide 1002 may be deformable to allow for a small amount of reverse pressure to be formed at a lower opening thereof (not shown) which may be transmitted direct to overbandage 1004 or through one or more holes in integrated support 1012 (if a support is used). In an embodiment, additional markings, colors, etc. on placement guide 1002, overbandage 1004, primary bandage/patch 1008, on-skin monitoring device 1006, and/or support 1012 may be used to orient and/or properly align the components.

In an embodiment, placement guide 1002 may comprise a housing having a base integrated with or coupled to overbandage support 1012. To accommodate placement of overbandage 1004 on/around on-skin monitoring device 1006, the base of the placement guide housing and/or support 1012 may have an opening (not shown). In this configuration, in an embodiment, overbandage 1004 may have material spanning the opening of overbandage 1004 as the material may be accommodated by the opening in the base of the placement guide housing and/or support 1012. In other embodiments, the opening of overbandage 1004 may be absent any such material.

FIG. 10A also shows overbandage 1004 with release liner 1014. Release liner 1014 may be removed from overbandage 1004 at any point during the placement process. In an embodiment, release liner 1014 may be removed after overbandage 1004 has been placed on/around on-skin monitoring device 1006 so that the adhesive surface of overbandage 1004 has less opportunity to contact dirt, debris, water, etc. or to accidentally adhere to the surface of on-skin monitoring device 1006.

FIG. 10B illustrates placement guide 1002 and support 1012 associated with overbandage 1004 after placement over an on-skin monitoring device. FIG. 10C illustrates overbandage 1004 having been placed on/around monitoring device 1006 and primary bandage/patch 1008, and shows the disengagement of placement guide 1002 and support 1012 from on-skin monitoring device 1006 and overbandage 1004.

In an embodiment, placement guide 1002 and support 1012 may be reused a plurality of times, or, if desired, may be disposable.

In an embodiment, there is provided an overbandage placement guide, comprising a housing having a base, an overbandage support coupled to the base and configured to couple with an overbandage for placement of the overbandage on and/or around a component on skin of a user, and the base and overbandage support having an opening configured to mate with an on-skin monitoring device, and configured to correspond to an opening of an overbandage to guide the orientation and/or direction of the overbandage on and/or around a component on the skin of the user.

Although certain embodiments have been illustrated and described herein for purposes of description of preferred embodiments, it will be appreciated by those of ordinary skill in the art that a wide variety of alternate and/or equivalent embodiments or implementations calculated to achieve the same purposes may be substituted for the embodiments shown and described without departing from the scope of the present invention. Those with skill in the art will readily appreciate that embodiments may be implemented in a very wide variety of ways. This application is intended to cover any adaptations or variations of the embodiments discussed herein. Therefore, it is manifestly intended that embodiments in accordance with the present invention be limited only by the claims and the equivalents thereof.

What is claimed is:

1. An overbandage, comprising:
a frame having an upper surface, a lower surface, an outer boundary and an inner boundary, wherein said inner boundary defines an interior opening configured to receive a monitoring device;
adhesive disposed on said lower surface of said frame; and
a release liner removably coupled to said adhesive.

2. The overbandage of claim 1, further comprising a material spanning said interior opening.

3. The overbandage of claim 2, wherein said material is transparent.

4. The overbandage of claim 2, wherein said material is waterproof.

5. The overbandage of claim 2, wherein said material is resistant to penetration by microorganisms.

6. The overbandage of claim 2, wherein said material is permeable to water vapor.

7. The overbandage of claim 2, wherein said material is flexible.

8. The overbandage of claim 2, wherein said material is free of adhesive at least in an area defined by said interior opening of said frame.

9. The overbandage of claim 2, wherein said material spanning said interior opening is disposed on said upper surface of said frame.

10. The overbandage of claim 9, further comprising an additional frame disposed on said material spanning said interior opening on said upper surface of said frame.

11. The overbandage of claim 1, wherein said release liner comprises multiple parts each removably coupled to said adhesive.

12. The overbandage of claim 11, wherein at least two of said multiple parts of said release liner overlap.

13. The overbandage of claim 11, wherein at least one of said multiple parts is folded.

14. The overbandage of claim 11, wherein said multiple parts comprise tabs for grasping and removing said release liner.

15. The overbandage of claim 14, wherein said tabs are at least one of color coded or numbered to indicate a recommended order of removal.

16. The overbandage of claim 1, wherein said frame comprises a plastic, foam, or fabric material.

17. The overbandage of claim 1, wherein said frame comprises a breathable material.

18. The overbandage of claim 17, wherein the breathable material comprises a plurality of pores.

19. The overbandage of claim 1, further comprising at least one wicking layer disposed on at least one of the upper surface of the frame and the lower surface of the frame between the lower surface and the adhesive.

20. The overbandage of claim 1, further comprising at least one wicking layer disposed on the upper surface of the frame and at least one breathable film disposed on said at least one wicking layer and forming an outermost surface of the overbandage.

21. The overbandage of claim 1, wherein said frame comprises a wicking material.

22. The overbandage of claim 21, further comprising at least one breathable film disposed on said frame and forming an outermost surface of the overbandage

23. The overbandage of claim 1, wherein said interior opening is configured to receive an on-skin portion of a continuous glucose monitoring device.

24. A method of further securing a monitoring device to skin, comprising:
placing an overbandage over and around the monitoring device, the monitoring device being already at least partially secured to the skin with a primary bandage, the overbandage comprising
a frame having an upper surface, a lower surface, an outer boundary and an inner boundary, wherein said inner boundary defines an interior opening;
adhesive disposed on said lower surface of said frame; and
a release liner removably coupled to said adhesive; and
removing the release liner to expose the adhesive and adhere the overbandage to the skin and over at least part of the primary bandage.

25. The method of claim 24, wherein said release liner comprises at least a first part and a second part each removably coupled to said adhesive, and wherein said removing the release liner comprises first removing said first part of said release liner and then removing said second part of said release liner.

26. An overbandage guide, comprising:
a housing having an opening configured to mate with an on-skin monitoring device, the housing having a base; and
the base having a size and/or shape configured to correspond to an opening of an overbandage to guide the orientation of the overbandage over the housing and onto skin of a user and on/around a component on the skin of the user.

27. An overbandage placement guide, comprising:
a housing having a base;
an overbandage support coupled to the base and configured to couple with an overbandage for placement of the overbandage on and/or around a component on skin of a user; and
the base and overbandage support having an opening configured to mate with an on-skin monitoring device, and configured to correspond to an opening of an overbandage to guide the orientation of the overbandage on and/or around a component on the skin of the user.
